(19) United States
(12) Patent Application Publication
(10) Pub. No.: US 2008/0199512 A1
(43) Pub. Date: Aug. 21, 2008

(54) Oral Composition For Moisturizing Skin

(76) Inventor: Steffi Dudek, Edgewater, NJ (US)

Correspondence Address: MORGAN & FINNEGAN, L.L.P., 3 WORLD FINANCIAL CENTER, NEW YORK, NY, 10281-2101, US

(21) Appl. No.: 12/065,459

(22) PCT Filed: Aug. 30, 2006
(86) PCT No.: PCT/NL2006/050211

§ 371 Date: Apr. 2, 2008

(30) Foreign Application Priority Data
Sep. 2, 2005 (EP) 05108057.0

(51) Int. Cl. A61K 9/68; A61K 31/70; A61K 9/00

(52) U.S. Cl. 424/440; 514/62; 424/439

(57) ABSTRACT

Moisture of the skin, skin lipid balance and skin smoothness can be improved by administering an oral composition containing, as active ingredients, proteins and/or protein hydrolysates wherein the total proteins and hydrolysates contain at least 2 wt. % of cysteine residues. Preferably, the hydrolysates have an average molecular weight of between 200 and 4000 Dalton. The composition is administered in such an amount that between 25 and 500 mg of cysteine per day is supplied.

The invention pertains to methods and products for maintaining or improving skin moisture and lipid conditions and skin smoothness.

### BACKGROUND

The most obvious function of the skin is to form a barrier to hold water inside the body, and to prevent penetration of water and environmental toxins and microbial invasions from outside. The skin is made up of three distinct layers.

The top layer is called the epidermis. The epidermis does not contain any blood vessels, and nutrient and oxygen supply is maintained from deeper layers of the skin. The basement membrane at the bottom of the epidermis attaches the epidermis firmly, though not rigidly, to the layer below. The second layer lies deeper and is called the dermis. It contains blood vessels, nerves, hair roots and sweat glands. Below the dermis lies a layer of fat, the subcutaneous fat.

All the cells in the epidermis originate ultimately from a single layer of basal cells, called the basal layer, which sits on the basement membrane. The ‘daughter cells’ produced by this basal layer gradually move upwards, lose their central nucleus, and start to produce skin proteins called keratins and fats called lipids. They are now known as keratinocytes, the process of skin cell transformation is called keratinisation. As they move upwards through the skin thickness their form slowly changes. The altered cells form distinct layers, which naturally blend into each other. The outermost layer of the epidermis, the horny “bark” of the skin, is called stratum corneum. The structure of the stratum corneum is essential to maintain the barrier function of the skin. The cells of this layer are continuously worn away and replaced from below with new cells. The wearing process is called desquamation.

Normal keratinisation and desquamation depend on timing and balance. A healthy stratum corneum is produced when cells are manufactured at a normal rate, when cells are shed at a normal rate, and when these two processes are in sync with each other.

If keratinocytes are produced faster than they can possibly be sloughed off, as is the case in psoriasis, scaly plaques form. If desquamation takes place too early, the skin isn't an effective protective barrier.

Healthy, attractive skin is directly related to optimal barrier function of the epidermis, especially of the stratum corneum. The loss of barrier function or an abnormal barrier function results in dry, brittle skin and increased susceptibility to infection and irritation. A major sign of a defective skin is the dryness that results from excessive water loss.

The presence of an adequate amount of water and epidermal lipids, in particular sphingolipids (or ceramides), fatty acids and cholesterol in the epidermis is not only important for the healthy skin to fulfil its barrier function, but also for the general appearance of a soft and smooth skin.

The thickness of the epidermis, especially of the stratum corneum, is another factor that affects the appearance of the skin. Thickening of the epidermis may be perceived as decreased smoothness and dullness of the skin, and may be accompanied with a feeling of the skin being less hydrated. The term “extrinsic aging” is used to describe this thickening of the epidermis, which is caused by exogenous factors, mainly photodamage.

Exogenous as well as endogenous factors are known to affect skin characteristics. The vast majority of products proposed thus far for improving skin characteristics are topical products, which focus on the exogenous factors. Such products include: (a) occlusives, which physically block water loss (examples: petrolatum and other oils and minerals); (b) humectants, which attract water to the stratum corneum (example: glycerin, sorbitol, urea); (c) emollients, which fill spaces between skin flakes (examples: cholesterol, squalene, fatty acids); and (d) rejuvenators, which are claimed to replenish essential proteins (examples: collagen, keratin, elastin). Efficacy of these topical compositions has been proven in only a few cases.

Interest in the role of endogenous factors on the skin barrier function and skin appearance has increased. Since endogenous factors are affected by nutrition, the role of nutrition for the skin appearance has been subject of studies for many years, but systematic data are limited. In a Dutch study, the impact of nutrient concentrations in serum and diet on skin conditions was studied (Boelsma et al., Am J. Clin. Nutr. 77 (2):348-355, 2003), with emphasis on vitamins and carotenoids.

Oral supplements for maintaining a healthy skin were only recently introduced. Most of them contain mixtures of antioxidants, and sometimes glucosamine, hyaluronic acid, ceramides or unsaturated fatty acids. Effects mainly concern the stress response on UV radiation and the restoration of the barrier function after injury (Thiele et al., Curr. Probl. Dermatol. 29: 26-42, 2001). Those ingredients are known either to prevent lipid peroxidation by strengthening the antioxidant defence in the epidermis, or to support the integrity of the extracellular hydrolipid matrix surrounding the epidermal cells, or to stimulate the immune activity in the skin.

It has to be emphasised that antioxidants, like carotenoids and high dose vitamins, may also exert a pro-oxidant potential, which is especially pronounced if iron is supplemented simultaneously, or iron is released by cell injuries.

JP-A 2004-107238 discloses a kit for improving barrier function of the stratum corneum and for retaining moisture in the skin. The kit comprises a topical compositions containing olive oil, squalene, sea minerals, serine, isomerised sugars or trimethyl glycine, and an oral composition containing ceramide, hyaluronic acid, silk peptide, glucosamine, glycine, niacin or collagen.

The oral supplement Evelle® contains a mix of antioxidants (pycnogenol, vitamin C, vitamin E, blueberry extract and the carotene-resource tomato extract, selenium), biotin, zinc gluconate, hydrolysed collagen and glucosaminoglycans from salmon, and a natural silicate resource. The product was shown to improve signs of cutaneous ageing (D. Segger and F. Schönlau. J Dermatolog. Treat. 15 (4):222-226, 2004)

Imedeen®, a supplement containing cartilage polysaccharides derived from marine fish was supposed to exert a repairing effect on photo-aging, and hyper-pigmentation, in self-evaluation of skin condition, density measurements by ultrasound, trans-epidermal water loss and skin smoothness after one year supplementation (M. E. Kieffer and J. Efsen. J Eur. Acad. Dermatol. Venereol. 11 (2):129-136, 1998.). A severe aversive event with Imedeen was reported in 2005 (J. F. van Leeuwen, C. S. van der Hooft, L. E. Vos, M. W. Bekkenk, E. J van Zuuren, and B. H. Stricker, Ned.Tijdschr.Geneeskd. 149 (24):1353-1356, 2005)

### DESCRIPTION OF THE INVENTION

It has been found that peptides and proteins containing a high proportion of cysteine residues, when administered orally, are capable of moisturising the skin, especially the stratum corneum, and of restoring a epidermal lipid balance involving sphyngolipids and ceramides. In doing so, the peptides prevent deterioration of the barrier function of the epidermis and restore the barrier function and hence they provide a more smooth, pleasant and supple skin feeling. The effect on the water and lipid balance and of the barrier function of the epidermis is distinct from the effect on specific skin problems which find their origin in the dermis. For instance, discoloration of the skin is related to disturbance of pigment formation in the dermis, and pathological skin conditions like e.g. acne are often caused by inflammation of sebaceous gland, which are also located in the dermis. Up to now, no satisfactory solutions are available that solve the problem of an insufficiently hydrated epidermis. The treatment according to the invention can primarily be such as to prevent or cure a health problems of the skin, and thus be medical. It is also intended to be used where cosmetic problems are associated with the health problems, or to treat conditions which are of a cosmetic nature only.

The peptides and/or proteins to be used for moisturising the skin according to the invention contain at least 2 wt. %, preferably at least 3 wt. % of cysteine residues, more preferably at least 5 wt. %, even more preferably at least 6 wt. % and most preferably at least 6.5% wt. %. The cysteine content of the peptides may be as high as e.g. 20 wt. %, but for practical purposes a cysteine content of up to 10 wt. % is sufficient. It is furthermore preferred that at least 50%, more preferably at least 70% of the peptides contain at least one terminal cysteine residue. In determining the cysteine content, the oxidation state of cysteine is disregarded, i.e. cystine residues are counted as two cysteine residues. The cysteine residues may also be in another equivalent form, such as thio-esters, which may be processed into free cysteine residues.

According to the invention, a single peptide or protein having the required cysteine content may be used. Often, it will be preferred to use a mixture of peptides and proteins. Peptides having a relatively high cysteine content may be used in combination with peptides or proteins having a lower cysteine content, as long as the total proteinaceous matter has a minimum cysteine content of 2 wt. %, preferably at least 3 wt. %, most preferably at least 5 wt. %. The peptide may be a protein having a molecular weight of e.g. up to 40,000 Da, preferably below 25,000 Da, and having a minimum cysteine content of 2 wt. %, or a mixture of such a protein with other proteins and/or peptides, as long as the total proteinaceous matter contains at least 2 wt. % of cysteine. In the present context, the terms ‘peptides’ and ‘proteins’ are used interchangeably, unless indicated otherwise.

It is preferred that the molecular weight of at least 70%, more preferably at least 80% (weight) of the peptides and proteins is smaller than 10 kDa, that means they contain less than 100 amino acids.

It is preferred that the content of free amino acids is less than 5 wt. %, although for food applications higher levels may sometimes be used, e.g. up to 10 wt. %. For safety reasons, it is preferred that the cysteines are in the oxidised (disulfide bridged) form. Especially, at least 80% of the cysteines is in the oxidised (cystine) form.

In a preferred embodiment, the cysteine-containing peptides and proteins comprise a mixture of peptides obtained by specific hydrolysis of cysteine-containing proteins and optional fractionation. The cysteine-containing peptides preferably have a weight-average molecular weight between 200 and 4000, more preferably between 300 and 2000 Da, most preferably between 500 and 1500 Da. In a specific embodiment, the peptide may also be or contain glutathione.

Suitable cysteine-containing proteins to be used as such or for being hydrolysed to cysteine-containing peptides include milk proteins, especially whey proteins, with a preference for whey protein concentrates (WPC 60 or WPC 80) or whey protein isolates. If available on economic scale, alpha-lactalbumin, beta-lactoglobulin or serum albumin may also be used. Other suitable protein sources include egg protein, wheat, maize, or rapeseed albumin protein, yeast or yeast extract.

The hydrolysis can be performed e.g. by first cleaving, enzymatically or chemically, the starting protein into a peptide mixture, followed by treatment of the peptide mixture with an exopeptidase which is relatively inactive in cleaving terminal cysteine residues. The prior cleavage can be performed by acid hydrolysis, but more preferably by endopeptidase treatment. Suitable endopeptidases include pepsine, Alcalase (Novo) and the like. Suitable exopeptidases include e.g. carboxypeptidase Y. The cleavage steps can also be carried simultaneously using a suitable mixture of endopeptidase and exopeptidase. Alternatively, and preferably, the hydrolysis can be performed in a single enzymatic step using an endopeptidase that also has a suitable exopeptidase activity. Examples of such enzymes having both endopeptidase and exopeptidase activity include Flavourzyme, Acid Protease A, Protease M, Protease 2A, Protease B, Acid Protease (EDC), and Corolase. Combinations of enzymes can also be used effectively.

The enzymatic reaction is carried out using the conditions which are appropriate for the particular enzyme or enzyme combination. The reaction conditions thus may comprise temperatures between 20 and 60° C., depending on the optimum temperature of the enzymes, pH values e.g. between 3 and 10, depending on the optimum pH of the enzymes, and reaction times that may vary between about 30 min. and 24 h. The reaction times are selected depending on the reaction temperature, and on the concentration, activity and specificity of the enzymes.

The hydrolysates obtained can be subjected to further separation using filtration (ultra, nano), chromatography (affinity, ion exchange or other), electrophoresis, extraction, precipitation, and other techniques known in the art, and combinations of such techniques. The final hydrolysate product may be “polished” by filtering over a filter aid, e.g. diatomaceous earth, to remove any insoluble matter. Very suitable methods of hydrolysing proteins for producing the cysteine-containing peptides are described in EP-A 1.201.137.

The peptide or peptides may also be obtained using chemical peptide synthesis or by means of recombinant DNA techniques.

The cysteine-containing peptides are administered for treating or preventing a hampered water and/or lipid balance and barrier function and of the epidermis, especially the stratum corneum and for providing a softer, more supple and pleasant skin feeling. They can be administered at a level which corresponds to the intake of 25 to 500 mg of cysteine per adult per day, or 0.3 to 8 mg per kg body weight per day. Preferred levels are from 50 to 250 mg per adult per day or from 0.7 to 4 mg per kg per day.

The cysteine-containing peptides can be administered as such or, preferably, as a pharmaceutical or nutritional composition. Pharmaceutical compositions include tablets, capsules, coated tablets, lozenges, syrups, powders, sachets, solutions, suspensions, jelly sachets. The pharmaceutical composition can further contain conventional excipients, such as lactose, carboxymethylcellulose, microcrystalline cellulose, silicon dioxide, pectin, chitosan, agar or alginic acid, stabilisers, flavours, colorants and the like. A pharmaceutical composition can comprise the daily dosage level in one dosage unit or in multiple (e.g. 2-6) daily dosage units). The total weight of the pharmaceutical composition may be in the range of 100-1000 mg of a dry form, e.g. 400-800 mg in case of a tablet. For the purpose of the invention, pharmaceutical compositions comprise cosmetic compositions.

Nutritional compositions include solid, semi-solid or liquid food products. Solid products comprise e.g. nutrition bars, (with protein, chocolate, muesli, nuts, etc.), cookies, sweets, chewing gums (e.g. containing 0.1-2.5 wt. % of cysteine in the form of peptides in addition to 10-40 wt. % of gum base, and further constituents), bread; semi-solid foods comprise ready made meals, cheese products, meat products, tofu, pasta. Liquid food products comprise beverages (e.g. soft drinks), milk or fermented milk products, like yoghurt, or drinking yoghurt, and the like. Optionally, stabilizers (pectin, carragheenan, locust bean gum, xanthan gum or other gums), emulsifiers (mono- and diglycerides, TWEEN, SPAN, sucrose esters of fatty acids), thickeners (starch, modified starch, maltodextrins, alginic acid, chitosan or salts thereof, other hydro-colloids) can be included in the nutritional products, as well as flavours and colorants. The thickeners, e.g. at a total weight level of 0.05-5 wt. % are especially useful for jelly-like products, such as jelly sachets. Food products containing the peptides of the invention, as well as their mixtures or mixtures with other peptides or other active components, may contain between 5 and 500 mg of cysteine per 100 g dry food, or per litre of liquid food. Supplements may contain higher levels of cysteine-containing peptides, e.g. between 0.1 and 2.5 wt. % cysteine on he basis of the supplement. Suitable supplements are e.g. tablets and powders for instant drinks.

The cysteine peptides can be combined with other active components or adjuvants for moisturising the skin, such as ceramides, hyaluronic acid, glucosamines, niacin, vitamins (C, E), carotenoids or other antioxidants and the like, particular in a weight ratio between cysteine-containing peptide and such other component of 9:1 to 1:4, especially between 4:1 and 1:2. The invention also concerns a pharmaceutical product containing a cysteine-containing peptide as described herein together with such additional component, especially glucosamine, N-acetyl-glucosamines, their hetero- and homo-, oligo- and polymers, including hyaluronic acid (poly[D-glucuronic acid-β-1,3-N-acetylglucoseamine-β-1,4-]) or ceramides ((N—C₁₂-C₂₀-acyl)-2-amino-C₁₂-C₂₀-alkane(or -alkene)-1,3-diols or -1,3,4-diols), preferably in the ratios as given above. In term of amounts of cysteine, the weight ratio between cysteine and the other one or components is preferably between 1:1 and 1:100, especially between 1:3 and 1:30.

### EXAMPLES

### Example I

### Preparation and ccharacteristics of a Protein Hydrolysate Enriched in Cysteine-Containing Peptides

A 10% (w/w) WPI (Bipro) was prepared and hydrolysed with 0.5% (w/w on substrate) pepsin (Merck) at pH 2.0, room temperature, for 6 hours. Next, the pH was increased to pH 7.0 using sodium hydroxide, and 0.5% (w/w on substrate) of Acid Protease (EDC) was added and the incubation was continued for 20 hrs (50° C.). The reaction was terminated by heating the solution to 85° C. for 15 minutes. Hereafter, the majority of the free amino acids was removed using ultrafiltration. A membrane with a nominal molecular cut-off of 1000 Dalton was used. The solution was diafiltered to 500% diafiltration. The retentate obtained was then spray-dried. Protein content of the end product was determined using the Kjeldahl method; cysteine content of the product was determined using Elmann's reagent (Beveridge et al., J. Food Sc. (1974) vol. 39, p 49-51). The molecular weight profile was determined using HPLC size exclusion chromatography (Shodex column).

The composition of the hydrolysate is as follows (w/w %)

| | |
|---|---|
| Protein | 86.3%. |
| Ash | 5.3% |
| Moisture | 4.7% |
| Cysteine/protein (w/w) | 7.5% |

The molecular weight distribution( HPLC) is as follows:

| Mol. weight (Da) | Average % (weight) |
|---|---|
| >10,000 | 18.5 |
| 10,000-5,000 | 3.9 |
| 5,000-2,000 | 6.7 |
| 2,000-1000 | 11.8 |
| 1,000-500 | 19.6 |
| <500 | 39.5 |

The product has a good solubility over a broad pH range. A 1% (w/w) solution in water shows a protein (nitrogen) solubility of at least 70% at low pH (3-5), and at least 80% at a pH 6-9.

Thermal stability: at moderate low pH, e.g. 3.5-4.0, the product remains stable (does not precipitate or flocculate) upon pasteurisation conditions (10-30 seconds at 65°-85° C.). At neutral pH, the product is also stable after sterilisation or UHT treatment.

### Example II

### Preparation of Capsules Containing Cysteine-Enriched Peptides

| Ingredients | weight % |
|---|---|
| Cysteine containing peptide | 95 |
| as described in example I | |
| Sugar ester (DK ester F20W, | 4 |
| Daiich Kogyo Seiyaku (Japan) | |
| Silicon dioxide | 1 |

The ingredients were thoroughly mixed and automatically filled in gelatin capsules. Total capsule weight: 280 mg. 3 capsules supplies 50 mg cysteine.

### Example III

### Dermatological Measurements After Ingestion of Cysteine Peptide

Study design: Placebo-controlled double-blind trial (parallel-group study among 4 groups to compare efficacy between before starting intake and 6 weeks after starting intake). 22 Female Japanese subjects (28-45 years old) were divided into 4 groups. They were asked to take 3, 6 or 12 capsules of the test product/day or a placebo (280 mg capsules with 100% microcrystalline cellulose (MCC) which were made in a similar way as he cysteine peptide containing capsules). The daily supplementation corresponded to 50 mg (group A), 100 mg (group B) or 200 mg cysteine (group C), respectively, or no cysteine supplementation (placebo group). The test product consisted of capsules containing cysteine-enriched peptides, as described in example II. Dose and administration scheme were as follows:

| Treatment | | | |
|---|---|---|---|
| group | Test product | Placebo | Time of intake |
| Group A | 3 capsules | — | At breakfast (the first meal |
| | (1 package) | | of the day) |
| Group B | 6 capsules | — | At breakfast (the first meal |
| | (2 packages) | | of the day) |
| Group C | 12 capsules | — | At breakfast and supper with |
| | (4 packages) | | 2 divided portions |
| Group P | — | 6 capsules | At breakfast (the first meal |
| | | (2 packages) | of the day) |

A number of parameters were measures that assess objectively skin properties. These methods are state of the art for dermatological investigations. The measurements took place at start and after 6 weeks taking the capsules containing the cysteine enriched peptides or placebo.

Analysis of skin oil content:

Skin lipids (oil) measurement was performed at a point 2 cm above the eyebrow on the forehead using a Sebumeter (Integral Corporation).

Water content of the skin surface and membrane thickness of the stratum corneum: Measurement was performed at a point of 4 cm on the straight line starting from below the right ear lobe toward the right edge of the lips using an ASA-MI (AsahiBiomed Co., Ltd).

The tables below show the relative changes observed after 6 weeks:

| | Water content of the skin, |
|---|---|
| Test Group | Increase (%) after 6 weeks |
| 50 mg cysteine group (A) | +20.4 |
| 100 mg cysteine group (B) | +48.1 |
| Placebo group (P) | +5.1 |

| | Membrane thickness | Oil (lipid) |
|---|---|---|
| | of the skin, Change (%) | content of the skin, |
| Test group | after 6 weeks | Change (%) after 6 weeks |
| 50 mg cysteine | −2.2% | +94.0% |
| group (A) | | |
| 200 mg cysteine | +27.0% | +105.0% |
| group (C) | | |
| Placebo group | +1.6% | −50.1% |
| (P) | | |

### Example IV

### Skin Perception Study After Intake of Cysteine Enriched Peptides

The study was executed in parallel with the above mentioned dermatological study, but with a separate subject cohort. 128 Female Japanese subjects, age 22-35 years, were divided into four groups of 32 subjects each. The groups received either placebo or a defined amount cysteine enriched peptides in capsules made according to example II.

The three treatment groups were asked to take 3, 6 or 12 capsules/day, in order to achieve a daily supplementation with 50, 100 mg or 200 mg cysteine, respectively, as in example III. After the study period, the subjects were asked to fill in questionnaires, in which the perception of skin properties was assessed in a quantified manner (grade 1-5).

Significant and dose dependent improvements were reported with respect to the following questions:

“Having a feeling of the skin being more slippery than before”

“Having a feeling of the skin being richer in moisture than before”

“Having a feeling of the skin being smoother than before”

In addition, the suppleness of the skin was also assessed.

Trends for improvements were also reported for some questions assessing skin and face appearance in general (pimples, dullness, oily shine, stickiness).

The results are given below:

| | | | | 200 mg |
|---|---|---|---|---|
| | Placebo | 50 mg group | 100 mg group | group |
| | (P) | (A) | (B) | (C) |
| More slippery | 3.4 | 3.6 | 3.7 | 4.3 |
| Richer in moisture | 3.6 | 3.8 | 3.9 | 4.7 |
| Smoother | 3.5 | 3.4 | 3.8 | 4.5 |
| More supple | 3.5 | 3.6 | 3.8 | 4.1 |

### Example V

### Preparation of Tablets

| Ingredient | Amount per 100 g |
|---|---|
| Cysteine-rich peptides | 88.24 g |
| Microcrystalline cellulose¹ | 10.59 g |
| Silicon Dioxide² | 0.47 g |
| Magnesium stearate | 0.35 g |
| Stearic acid | 0.35 g |

¹Avicel PH-102 - FMC
²CAB-O-SIL M-5

The powders were premixed whilst the magnesium stearate was withheld for the last minutes of mixing. The tablets were prepared by direct compression (compression pressure 20 kN, hardness: 160 N). Tablet weight was 850 mg.

### Example VI

### Skin Moisturising Beverage

| Ingredient | g per 100 g | g per serving (250 ml) |
|---|---|---|
| Cysteine peptide | 0.43 | 1.08 |
| Cysteine content | 0.024 | 0.06 |
| Dextrose | 4.4 | 11 |
| Fructose | 3.6 | 9 |
| Flavour apple | 0.055 | 0.14 |
| Flavour banana | 0.037 | 0.09 |
| Pectin¹ | 0.15 | 0.37 |
| Water | 91.3 | 228.4 |

¹Genu Pectine YM-115-H CP Kelco

All dry ingredients were dissolved in water and the pH was adjusted first till pH 3.8 with citric acid (±0.14% on total), than till pH 3.5 with malic acid (±0.66% on total). The solution was preheated to 70° C. followed by addition of the pectin premix (4% in water). After homogenisation (150 bar) the product was filled and pasteurised or pasteurised and filled aseptically.

1. (canceled)

2. (canceled)

3. (canceled)

4. (canceled)

5. (canceled)

6. (canceled)

7. (canceled)

8. A nutritional or pharmaceutical composition containing one or more peptides and/or proteins having a total cysteine content at least 2 wt. %, together with at least one active component selected from glucosamines and derivatives thereof, ceramides, niacin, vitamins, carotenoids and other antioxidants, especially glucosamines, hyaluronic acid and ceramides.

9. A composition according to claim 8, which is a dietary supplement, typically a pill or capsule, or a functional food product, typically a jelly sachet, a refreshing drink, a yoghurt or yoghurt drink, a milk based drink, an instant drink or any other functional drink, a chewing gum, candy, candy bars or the like.

10. A nutritional or pharmaceutical composition containing one or more peptides and/or proteins having a total cysteine content at least 2 wt. %, which is a jelly sachet, a refreshing drink, a yoghurt, a yoghurt drink or other milk-based drink, a chewing gum, candy or a candy bar.

11. A method for restoring or supporting the skin barrier function, involving enhancing skin moisture, skin lipid balance and/or skin smoothness, comprising administering to a subject in need thereof, an effective amount of one or more peptides or proteins having a total cysteine content at least 2 wt. %.

12. The method according to claim 11, wherein the total cysteine content of said one or more peptides and/or proteins is at least 3 wt. %.

13. The method according to claim 12, wherein the total cysteine content of said one or more peptides and/or proteins is between 5 and 20 wt. %.

14. The method according to claim 11, wherein said one or more peptides and proteins have an average molecular weight of between 200 and 4000 Dalton.

15. The method according to claim 11, wherein at least 70% of the peptides of the mixture has a molecular weight of lower than 10 kDa.

16. The method according to claim 11, wherein the composition is to be administered at a dosage corresponding to between 5 and 250 mg of cysteine per day.

17. The method according to claim 11, wherein the composition further contains other agents for improving skin conditions, selected from glucosamines, hyaluronic acid, ceramides, niacin, vitamins, carotenoids and other antioxidants.
